(19) United States
(12) Patent Application Publication
(10) Pub. No.: US 2019/0298301 A1
(43) Pub. Date: Oct. 3, 2019

(54) ULTRASOUND GUIDED POSITIONING OF THERAPEUTIC DEVICE

(71) Applicant: KONINKLIJKE PHILIPS N.V., EINDHOVEN (NL)

(72) Inventor: STEVE SUN, LEXINGTON, MA (US)

(21) Appl. No.: 16/468,154

(22) PCT Filed: Dec. 8, 2017
(86) PCT No.: PCT/EP2017/081936

§ 371 Date: Jun. 10, 2019

Related U.S. Application Data
(60) Provisional application No. 62/433,069, filed on Dec. 12, 2016.

(51) Int. Cl. A61B 8/08; A61B 5/06; A61B 34/20

(52) U.S. Cl. CPC A61B 8/0841; A61B 2017/3413; A61B 34/20; A61B 5/063

(57) ABSTRACT

An apparatus for performing a medical procedure comprises a sensor adapted to convert an ultrasonic signal incident thereon into an electrical signal. The sensor comprises a lower electrode and an upper electrode, and the upper electrode is adapted to transmit the electrical signal to an electrode of an ultrasound imaging probe.

### BACKGROUND

Location tracking of medical devices used in-situ on a patient enables less invasive medical procedures to be carried out. By way of example, ultrasound-guided medical procedures enable the location of certain medical devices relative to a position of interest in a patient.

In certain ultrasound based medical device tracking, electrical wires running from the tip to the handle of the medical device transmit signals to a console/workstation for data analysis.

Among other drawbacks, the connection of the medical instrument to the console/workstation by cables complicates clinical workflow, and introduces undesirable cable management. As a result, the clinical workflow is often impeded because of the presence of cables connecting the medical device to the console. This not only makes it cumbersome for the clinician to perform the procedure, but also limits the market acceptance of such known cable-connected devices and systems.

Accordingly, it is desirable to provide an apparatus, systems, methods, and computer-readable storage media for determining a position of a medical instrument, in-situ, which overcomes at least the short-comings of the above-described known devices.

### BRIEF DESCRIPTION OF THE DRAWINGS

The present invention will be more readily understood from the detailed description of representative embodiments presented below considered in conjunction with the accompanying drawings, as follows.

FIG. 1A is a conceptual diagram depicting two-way ultrasound signal transmission, in accordance with a representative embodiment.

FIG. 1B is a conceptual diagram depicting one-way ultrasound signal transmission, in accordance with a representative embodiment.

FIG. 2 is a schematic block diagram showing an ultrasound system, in accordance with a representative embodiment.

FIG. 3 is a simplified schematic block diagram showing a medical device, in accordance with a representative embodiment.

FIG. 4 is a graphical representation of electrical reactance versus electrical impedance of human tissue.

FIG. 5A is a conceptual diagram depicting a frame scan using a plurality of ultrasound beams.

FIG. 5B shows the relative timing of frame trigger signals, line trigger signals, and a received sensor signal of a medical device in accordance with a representative embodiment.

### DETAILED DESCRIPTION

The present teachings are described hereinafter with reference to the accompanying drawings, in which representative embodiments are shown. The present teachings may, however, be embodied in different forms and should not be construed as limited to the embodiments set forth herein. Rather, these embodiments are provided as teaching examples.

Generally, according to various embodiments, is to be understood that the terminology used herein is for purposes of describing particular embodiments only, and is not intended to be limiting. Any defined terms are in addition to the technical and scientific meanings of the defined terms as commonly understood and accepted in the technical field of the present teachings.

As used in the specification and appended claims, the terms “a”, “an” and “the” include both singular and plural referents, unless the context clearly dictates otherwise. Thus, for example, “a device” includes one device and plural devices.

Unless otherwise noted, when an element or component is said to be “connected to,” “coupled to” another element or component, it will be understood that the element or component can be directly connected, directly coupled to the other element or component, or, intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

Also, it will be understood that, in addition to their ordinary meanings, the terms “substantial” or “substantially” mean to within acceptable limits or degree to one having ordinary skill in the art. For example, “substantially cancelled” means that one of ordinary skill in the art would consider the cancellation to be acceptable. Likewise, in addition to its ordinary meaning, the term “approximately” means to within an acceptable limit or amount to one having ordinary skill in the art. For example, “approximately the same” means that one of ordinary skill in the art would consider the items being compared to be the same.

Directional terms/phrases and relative terms/phrases may be used to describe the various elements' relationships to one another, as illustrated in the accompanying drawings. These terms/phrases are intended to encompass different orientations of the device and/or elements in addition to the orientation depicted in the drawings.

Like numbered elements in these figures are either equivalent elements or perform the same function. Elements which have been discussed previously will not necessarily be discussed in later figures if the function is equivalent.

Initially, it is noted that medical images may include 2D or 3D images such as those obtained using an ultrasound imaging probe, and a position of a medical instrument relative to an image frame of ultrasound signals from the ultrasound imaging probe.

In accordance with a representative embodiment, an apparatus for performing a medical procedure comprises a sensor adapted to convert an ultrasonic signal incident thereon into an electrical signal. The sensor comprises a lower electrode and an upper electrode, and the upper electrode is adapted to transmit the electrical signal to an electrode of an ultrasound imaging probe.

In accordance with another representative embodiment, an ultrasound system, comprises: an ultrasound imaging probe adapted to insonify a region of interest; an apparatus configured to perform a medical procedure. The apparatus comprises: a sensor adapted to convert an ultrasonic signal incident thereon into an electrical signal. The sensor comprises a lower electrode and an upper electrode, wherein the upper electrode is adapted to wirelessly transmit the electrical signal to an electrode of the ultrasound imaging probe. The ultrasound system also comprises a control unit remote from the ultrasound imaging probe and apparatus, the control unit being adapted to provide an image from the ultrasound imaging probe. The control unit comprises a processor adapted overlay the a position of the apparatus on the image.

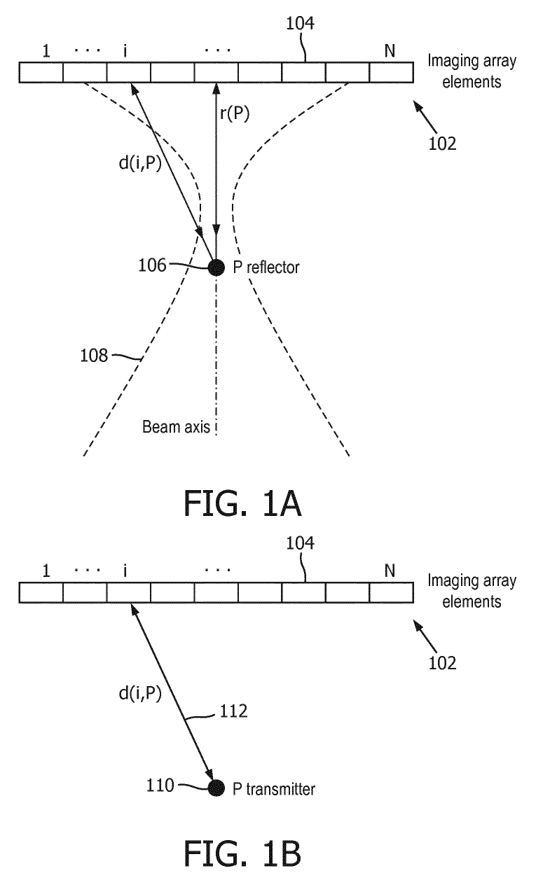

FIGS. 1A and 1B offer, by way of an illustrative and non-limitative example, a comparison between two-way beamforming (FIG. 1A) and one-way only beamforming (FIG. 1B).

Turning to FIG. 1A, representative of two-way beamforming shows an imaging array 102 of N elements 104 issuing ultrasound signals that impinge on a reflector 106. Since the ultrasound waves go out and back (from the imaging array to the reflectors and back to the imaging array), this beamforming is “two-way” or “round-trip” beamforming. On receive (of the ultrasound that has reflected back), beamforming determines the reflectivity of the reflector 106 and the position of the reflector relative to the array 102. The array 102 sends out an ultrasound beam 108 that is reflected from the reflector 106 and returns to all elements 104 of the array 102. The flight of the beam is over a distance r(P)+d(i,P) for element i. Each element 104 measures continually the amplitude of the return ultrasound. For each element 104, the time until a maximum of that measurement, i.e., the “round-trip time of flight,” is indicative of the total flight distance. Since the r(P) leg of the flight is constant, the return flight distance d(i,P) is determined. From these measurements, the relative position of the reflector 106 is computed geometrically. As to the reflectivity of the reflector 106, it can be indicated by summing the maxima over all i (i.e., over all elements 104).

Turning to FIG. 1B, one-way only (receive) beamforming is depicted. Notably, as the name implies, in one-way beamforming there is echo, but it is not used. Instead, an ultrasound transmitter 110 emits an ultrasound beam 112, which is incident on each element 104 of the array 102. The flight here, in contrast to the two-way beamforming case, is over the distance d (i,P). The time from emission of the ultrasound beam 112 until the maximum amplitude reading at an element 104 determines the value d (i,P) for that element i. Thus, the position of the ultrasound transmitter 110 can be derived geometrically, and the reflectivity calculated by summing the maximum amplitude readings.

Although one-way beamforming is implementable in the time domain via delay logic, as discussed hereinabove, it can also be implemented in the frequency domain by well-known Fourier beamforming algorithms.

As will become clearer as the present description continues, two-way beamforming is used to gather images on a frame-by-frame basis; and one-way beamforming is used to determine the location of a sensor disposed at or near a distal end of a medical device (sometimes referred to generically as an apparatus).

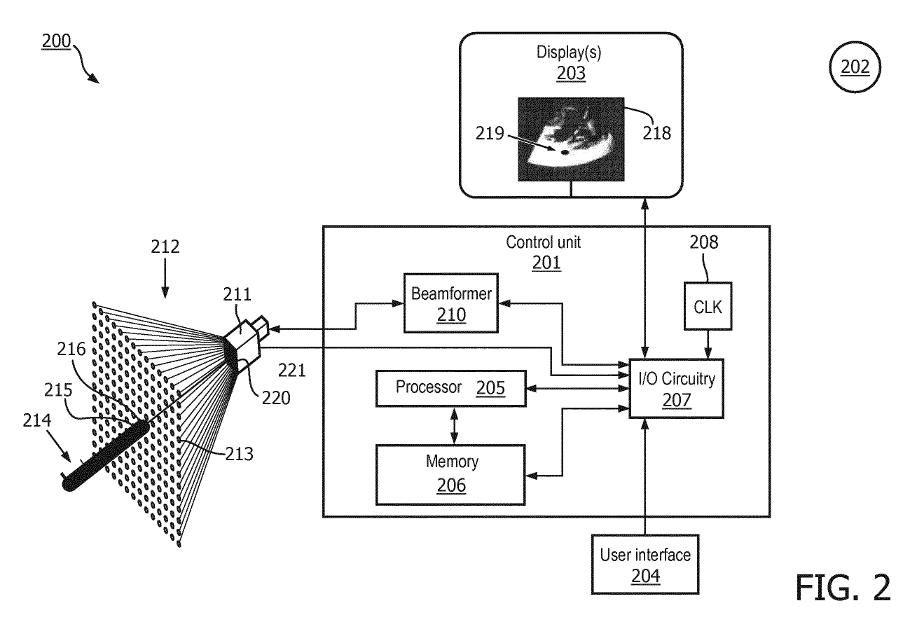

FIG. 2 is a simplified schematic block diagram showing an ultrasound system 200, in accordance with a representative embodiment of the present invention. The ultrasound system 200 comprises a number of components, the functions of which are described more fully below.

The ultrasound system 200 comprises a control unit 201, which is connected to a display 203, and a user interface 204. The control unit 201 comprises a processor 205, which is connected to a memory 206, and input output (I/O) circuitry 207. The ultrasound system 200 also comprises an ultrasound imaging probe 211 and a medical device 214.

The control unit 201 comprises a beamformer 210. The beamformer 210 is adapted to receive signals from the ultrasound imaging probe 211. The ultrasound imaging probe 211 is connected to hardware 212, (i.e. transducer hardware) which senses ultrasound for performing receive beamforming used in two-way (e.g., pulse-echo) imaging of the region of interest 213. As described more fully below, the ultrasound imaging probe 211 is adapted to scan the region of interest 213, and provides images, which are built digitally, line-by-line, on a frame-by-frame basis.

The control unit 201 further comprises a clock (CLK) 208 (sometimes referred to below as a first clock), which may be a component of a beamformer 210. The clock 209 provides clock signals, to the I/O circuitry for distribution to and use in the ultrasound system 200, as described more fully below. As will become clearer as the present description continues, the clock 208 is useful in determining a position of a medical device 214 in situ in a coordinate system of an image frame of an ultrasound imaging probe 211.

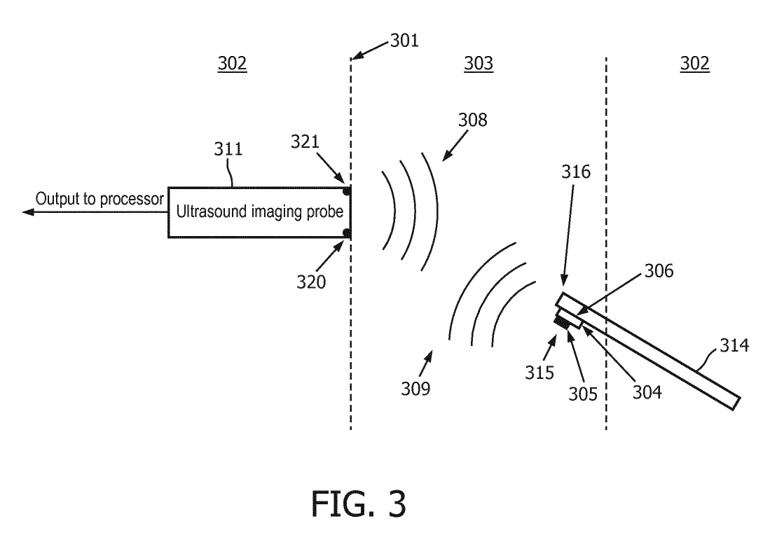

The medical device 214 comprises a sensor 215 (see FIG. 3) disposed at or near, (i.e. a known distance from) a distal end 216, which is disposed at a target location in the region of interest 213. As described more fully below, the sensor 215 is adapted to convert ultrasound beams provided by the ultrasound imaging probe 211 into electrical signals. These electrical signals are transmitted through the body and are incident on a sensing electrode 220 on the ultrasound imaging probe 211. The sensing electrode 220 provides these electrical signals through a link 221 to the I/O circuitry 207 for use by the processor 205 to determine a location of the sensor 215, and thereby distal end of the medical device 214 in the coordinate system of an image in a particular image frame.

As will become clearer as the present description continues, the control unit 201 is illustratively a computer system, which comprises a set of instructions that can be executed to cause the control unit 201 to perform any one or more of the methods or computer based functions disclosed herein. The control unit 201 may operate as a standalone device (e.g., as the computer of a stand-alone ultrasound system), or may be connected, for example, using a wireless network 202, to other computer systems or peripheral devices. Generally, connections to the network 202 are made using a hardware interface, which is generally a component of input/output circuitry, which is described below.

In accordance with various embodiments of the present disclosure, the methods described herein may be implemented using the hardware-based control unit 201 that executes software programs. Further, in a representative embodiment, implementations can include distributed processing, component/object distributed processing, and parallel processing. Virtual computer system processing can be constructed to implement one or more of the methods or functionality as described herein, and the processor 205 described herein may be used to support a virtual processing environment.

In accordance with a representative embodiment, the display 203 is an output device or a user interface adapted for displaying images or data. A display may output visual, audio, and or tactile data. The display 203 may be, but is not limited to: a computer monitor, a television screen, a touch screen, tactile electronic display, Braille screen, Cathode ray tube (CRT), Storage tube, Bistable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, and Head-mounted display.

The user interface 204 allows a clinician or other operator to interact with the control unit 201, and thereby with the ultrasound system 200. The user interface 204 may provide information or data to the operator and/or receive information or data from the clinician or other operator, and may enable input from the clinician or other operator to be received by the control unit 201 and may provide output to the user from the control unit 201. In other words, the user interface 204 may allow the clinician or other operator to control or manipulate the control unit, and may allow the control unit 201 to indicate the effects of the control or manipulation by the clinician or other operator. The display of data or information on the display 203 or graphical user interface is an example of providing information to an operator. The receiving of data through a touch screen, keyboard, mouse, trackball, touchpad, pointing stick, graphics tablet, joystick, gamepad, webcam, headset, gear sticks, steering wheel, wired glove, wireless remote control, and accelerometer are all examples of user interface components which enable the receiving of information or data from a user.

The user interface 204, like the display 203, are illustratively coupled to the control unit 201 via a hardware interface (not shown) and the I/O circuitry 207, as would be appreciated by those skilled in the art. The hardware interface enables the processor 205 to interact with various components of the ultrasound system 200, as well as control an external computing device (not shown) and/or apparatus. The hardware interface may allow the processor 205 to send control signals or instructions to various components of the ultrasound system 200, as well as an external computing device and/or apparatus. The hardware interface may also enable the processor 205 to exchange data with various components of the ultrasound system, as well as with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

In a networked deployment, the control unit 201 may operate in the capacity of a server or as a client user computer in a server-client user network environment, or as a peer control unit in a peer-to-peer (or distributed) network environment. The control unit 201 can also be implemented as or incorporated into various devices, such as a stationary computer, a mobile computer, a personal computer (PC), a laptop computer, a tablet computer, a wireless smart phone, a set-top box (STB), a personal digital assistant (PDA), a global positioning satellite (GPS) device, a communications device, a control system, a camera, a web appliance, a network router, switch or bridge, or any other machine capable of executing a set of instructions (sequential or otherwise) that specify actions to be taken by that machine. The control unit 201 can be incorporated as or in a particular device that in turn is in an integrated system that includes additional devices. In a representative embodiment, the control unit 201 can be implemented using electronic devices that provide voice, video or data communication. Further, while a single control unit 201 is illustrated, the term “system” shall also be taken to include any collection of systems or sub-systems that individually or jointly execute a set, or multiple sets, of instructions to perform one or more computer functions.

The processor 205 for the control unit 201 is tangible and non-transitory. As used herein, the term “non-transitory” is to be interpreted not as an eternal characteristic of a state, but as a characteristic of a state that will last for a period of time. The term “non-transitory” specifically disavows fleeting characteristics such as characteristics of a particular carrier wave or signal or other forms that exist only transitorily in any place at any time.

The processor 205 is an article of manufacture and/or a machine component. As described more fully below, the processor 205 is configured to execute software instructions in order to perform functions as described in the various representative embodiments herein. The processor 205 may be a general purpose processor or may be part of an application specific integrated circuit (ASIC). The processor 205 may also be a microprocessor, a microcomputer, a processor chip, a controller, a microcontroller, a digital signal processor (DSP), a state machine, or a programmable logic device. The processor 205 may also be a logical circuit, including a programmable logic device (PLD) such as a programmable gate array (PGA), a field programmable gate array (FPGA), or another type of circuit that includes discrete gate and/or transistor logic. The processor 205 may be a central processing unit (CPU), a graphics processing unit (GPU), or both. Additionally, the processor 205 may include multiple processors, parallel processors, or both. Multiple processors may be included in, or coupled to, a single device or multiple devices of the ultrasound system 200.

Alternatively, in accordance with a representative embodiment, and as alluded to above, dedicated hardware implementations, such as application-specific integrated circuits (ASICs), programmable logic arrays and other hardware components, can be constructed to implement one or more of the methods and processes described herein. One or more representative embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

The memory 206 is an article of manufacture and/or machine component, and is a computer-readable medium from which data and executable instructions can be read by a computer. The memory 206 may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

Generally, the memory 206 comprises a tangible storage medium that can store data and executable instructions, and are non-transitory during the time instructions are stored therein. Further, the instructions stored in memory 206, when executed by the processor 205, can be used to perform one or more of the methods and processes as described herein. In a particular embodiment, the instructions may reside completely, or at least partially, within the memory 206. Notably, the instructions may reside within the processor 205 during execution by the control unit 201.

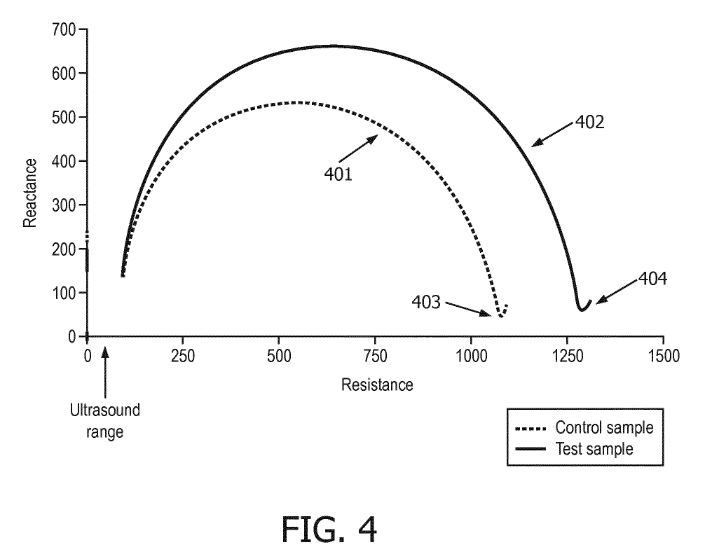

In accordance with a representative embodiment described below in connection with FIGS. 3, 4A and 4B, the position of the distal end 216 of the medical device 214 is determined by the processor 205 based on electrical signals from the sensor 215. To this end, the instructions stored in memory 206 are executed by the processor 205 to determine a position of the sensor 215 (and thus the distal end 216) in an image frame, and thus the distal end 216 of the medical device 214 in the coordinate system of the image of each frame. One illustrative method of determining the position of the distal end 216, for which instructions are stored in memory 206 is described below in connection with FIGS. 4A and 4B. Using the position of the sensor 215, the processor 205 executes instructions stored in memory 206 to overlay the position of the sensor 215 in an image frame, and thus the distal end 216 of the medical device 214 relative to the image of each frame.

The input/output (I/O) circuitry 207 receives inputs from various components of the ultrasound system 100, and provides output to and receives inputs from the processor 205, as is described more fully below. Input/output (I/O) circuitry 207 controls communication to elements and devices external to the control unit 201. The I/O circuitry 207 acts as an interface including necessary logic to interpret input and output signals or data to/from the processor 205. The I/O circuitry 207 is configured to receive the acquired live images from the beamformer 210, for example, via a wired or wireless connection. The I/O circuitry 207 is also configured to receive the electrical signals from the sensing electrode 220. As described more fully below, the I/O circuitry 207 provides these data to the processor 205 to ultimately superpose the location of the distal end 216 of the medical device 214 in a particular image frame.

Broadly, in operation, based on input from the user interface 204 provided to the processor 205 by the I/O circuitry 207, the processor 205 initiates a scan by the ultrasound imaging probe 211. The scan launches ultrasound waves across the region of interest 213. The ultrasound waves are used to form an image of a frame by the beamformer 210; and to determine the location of the sensor 215 of the medical device 214. As can be appreciated, the image is formed from a two-way ultrasound transmission sequence, with images of the region of interest being formed by the transmission and reflection of sub-beams by a plurality of transducers. By contrast, these sub-beams are incident on the sensor 215, which converts the ultrasound signals into electrical signals in a one-way ultrasound method. As described below in connection with FIGS. 4A and 4B, based on frame and line trigger signals provided to the ultrasound imaging probe 211, the location of the sensor 215 is determined.

A composite image 218, comprising the image of the frame from the ultrasound imaging probe 211 and the superposed position 219 of the sensor 215 in that frame is provided on the display 203 providing real-time feedback to a clinician of the position of the distal end 216 of the medical device 214 relative to the region of interest 213. As can be appreciated, the superposing of the position of the sensor 215 is repeated for each frame to enable complete real-time in-situ superposition of the location 219 of the sensor 215 relative to the image of the particular frame.

FIG. 3 is a simplified schematic block diagram showing a medical device 314 (sometimes referred to as an apparatus), in accordance with a representative embodiment. Many details of the medical devices described above in connection with FIGS. 1A-2 are common to the details of medical device 314, and may not be repeated in the description of the medical device 314.

The medical device 314 is contemplated to be any one of a number of medical devices where the location of a distal end is relative to a position in a region of interest, including but not limited to a needle, such as a biopsy or therapeutic needle, or a medical instrument, such as a laparoscope, or a scalpel. It is emphasized that the listed medical devices are merely illustrative, and other medical devices that benefit a clinician through the determination of their distal ends are contemplated.

Turning to FIG. 3, the medical device 314 comprises a sensor 315 disposed at, or at a known distance from, distal end 316. As described above, the sensor 315 is adapted to convert ultrasonic (mechanical) waves incident thereon into electrical signals. In a representative embodiment, the sensor 315 comprises a piezoelectric element 304, disposed between an upper electrode 305 and a lower electrode 306. Notably, if the sensor 315 is disposed in a portion of the medical device 314 that comprises an electrically conductive portion, the electrically conductive portion of the medical device 314 can function as the lower electrode.

In a representative embodiment, the piezoelectric element 304 may comprise a thin film piezoelectric material, such as lithium niobate (LiNbO₃), aluminum nitride (AlN), zinc oxide (ZnO), and lead-zirconate-titinate (PZT). Alternatively, the piezoelectric element 304 may comprise a piezoceramic material.

Upper electrode 305 and a lower electrode 306 may comprise any compatible electrically conductive material, such as molybdenum (Mo) or tungsten (W).

The lower electrode 306 is illustratively connected to electrical ground, and the upper electrode 305 serves as a transmitter. Upon incidence of an ultrasound signal, the sensor 315 effects the conversion of mechanical waves (energy) into electrical waves (energy), and the upper electrode 305 transmits the resultant electrical signal through a portion of a body 303, where the electrical signal is incident on the sensing electrode 320.

The distal end 316 of the medical device 314 is disposed in a body 303, such as the body of a person or other animal. An ultrasound imaging probe 311 is disposed at an interface of the body 303 (i.e., at a surface of the body 303) and the ambient 302. More simply, the ultrasound imaging probe 311, and especially the transducer array thereof (not shown) is in contact with the skin of the body 303, either directly or with a commonly used gel to improve any acoustic impedance mismatch between the transducer array and the body 303, and improve any electrical impedance mismatch between the sensing electrode(s) 320, 321 and the body 303.

The ultrasound imaging probe 311 comprises a sensing electrode 320 adapted to receive an electrical signal transmitted from the sensor 315 through the body 303, as described more fully below. The sensing electrode 320 may be a known electrocardiogram (ECG) electrode, or other electrode. Notably, in certain embodiments, if an ECG or similar electrode were used for the sensing electrode 320, such an electrode does not have to be disposed on the ultrasound imaging probe 311. As the electrical signals are transmitted from the sensor virtually the same time as the acoustic waves are sensed by piezoelectric element 304, the timing of the ultrasound signal will provide position information of piezoelectric element 304 irrespective of where sensing electrode 320 is placed. However, placing the sensing electrode 320 on the ultrasound imaging probe 311 provides convenience and operational simplicity to the user.

The sensing electrode 320 may be integrated into the transducer array of the ultrasound imaging probe 311, or may be disposed adjacent to the array of transducers of the ultrasound imaging probe 311. Notably, to ensure reception of an electrical signal from the sensor 315, the area of the sensing electrode 320 must be sufficiently large to capture enough electrical energy to provide a useful electrical signal to the control unit to determine a position of the distal end 316 in an ultrasound image frame.

In another representative embodiment, another sensing electrode 321 may be provided on a side opposite to the sensing electrode 320, and adjacent to the ultrasound transducer array of the ultrasound imaging probe 311. In yet other representative embodiments, more than two sensing electrodes 320, 321 can be provided.

In addition to improving the power of the received signal compared to having just one sensing electrode through the increased sensing area for receiving the electrical signals (e.g., electrical signals 309 describe below), the sensing electrode 321 also provides redundancy in the event that the sensing electrode 320 does not receive a suitably sufficient electrical signal.

As can be appreciated, any electrical signal that is conducted from an aqueous medium (e.g., the body 303) to a metallic conductor (e.g., sensing electrode(s) 320, 321) requires a redox pair such as Ag/AgCl included in a typical ECG electrode to complete the circuit effectively. Otherwise, there will be a double layer of unknown capacitance formed, which can introduce noise into the signal due to fluctuation of its electrical impedance. As such, in accordance with a representative embodiment, the sensing electrodes 320, 321 comprise a suitably redox pair to improve the signal-to-noise (SNR) ratio. By contrast, other materials may be used to provide an antenna function through the sensing electrode(s), although the SNR may be compromised to an unacceptable level.

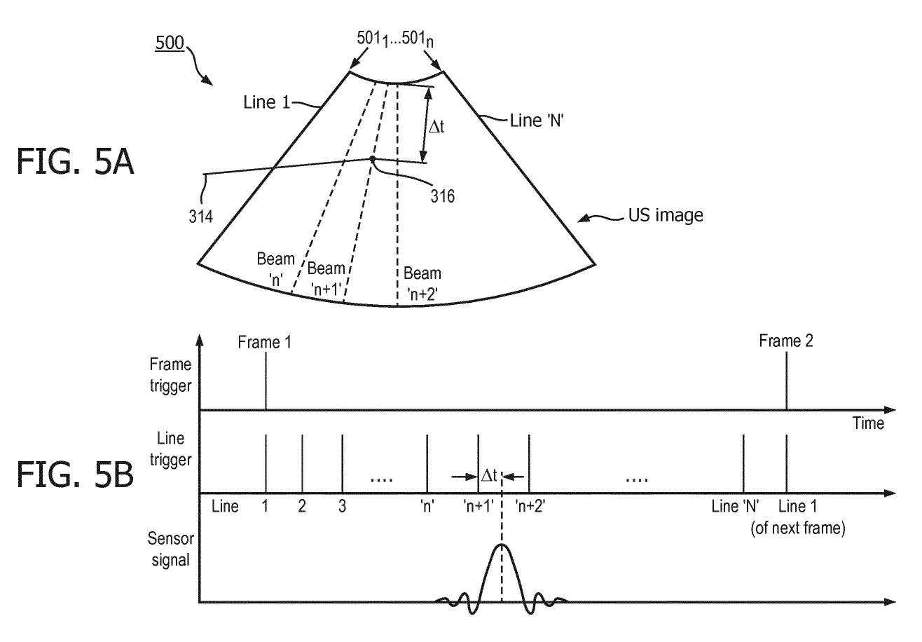

In operation, a frame trigger (see FIG. 5B) and line triggers (see FIG. 5B) cause excitation of the array of transducers in the ultrasound imaging probe 311, and ultrasound signals 308 are launched from the ultrasound imaging probe 311 into the body 303. The ultrasound signals 308 are incident on the sensor 315, which converts the ultrasound signals 308 into electrical signals 309. The electrical signals 309 are radiated from the upper electrode 305, which acts like a point source, through the body 303, and are incident on the sensing electrode(s) 320, 321. As can be appreciated, the electrical signals 309 are substantially synchronous with the electrical signals that excite the ultrasound transducers of the ultrasound imaging probe 311. These electrical signals 309 can thus be transmitted to the processor 205 of the control unit 201. In accordance with a representative embodiment, the electrical signals 309 are transmitted in a separate channel (e.g., link 221) from the channels of the ultrasound imaging probe 311. As can be appreciated, use of multiple channels increases the reliability of the sensor 315 through redundancy. Notably, however, not all the channels need to be functioning at the same time.

FIG. 4 is a graphical representation of electrical reactance versus electrical impedance of human tissue.

Turning to FIG. 4, the electrical impedance (Bioimpedance) of human tissues and organs are often described with Cole-Cole plot, such as in FIG. 4, which depicts the reactance of the tissue plotted against the resistance. Notably, the frequency of the electrical signals is omitted in the plot as the curve shifts from person to person. The frequency towards the origin where reactance is very small for virtually all people is where ultrasound frequencies are situated. Notably, curves 401 and 402 depict the reactance versus resistance of a test sample and a control sample of human tissue, respectively.

Curves 402 or 404 are physiologically relevant range of reactance versus resistance that clinicians can use bioimpedance to interpret the patient state in many areas. Curves 401 or 401 are in ranges with good signal to noise ratio that single frequency bioimpedance measurement can be made. Curves 404 or 403 are actually the limits that are seldom used alone other than within a complete spectral Cole-Cole Plot scan.

From a review of FIG. 4 it can be observed that ultrasound frequency radio wave generated by the sensor that converts mechanical wave to electromagnetic wave should travel through the body virtually unaffected other than the typical inverse square law on distance it travels. At ultrasound frequencies, both reactance and resistance of human tissue drop to this values. Body tissue become virtually transparent and ultrasound frequency radio waves will pass through body with ease. Notably, bioimpedance equipment are often not capable of deriving data below 100 Ω.

Typical human tissue becomes purely resistance at frequency greater than approximately 1.0 MHz. At such frequency range, the bioimpedance is very small and electrical signals freely propagate in the body while suffering little attenuation. Ultrasound impedance also falls in this frequency domain. Of course, the ultrasound wave is a mechanical wave, and does not interact with the electrical signal except in a region space where a transducer is present.

In accordance with representative embodiments, the ultrasound to electrical signal conversion provided by the sensor 315 results in electrical signals 309 beneficially having a frequency greater than approximately 1.0 MHz so the reactance is generally less than approximately 100 Ohms. Illustratively, the ultrasound signals 308 provided to the sensor 315 are converted to electrical signals 309 beneficially having a frequency so the reactance is in the range of approximately 0 Ohms to less than approximately 100 Ohms.

FIG. 5A is a conceptual diagram depicting a frame scan 500 using a plurality of ultrasound beams using an ultrasound system of a representative embodiment. FIG. 5B shows the relative timing of frame trigger signals, line trigger signals, and a received sensor signal of a medical device in accordance with a representative embodiment. Many details of the medical devices described above in connection with FIGS. 1A-3B are common to the details of the conceptual diagram and timing diagram of FIGS. 5A-5B, and may not be repeated in their description.

Turning to FIG. 5A, medical device 314 having sensor 315 at, or at a known distance from, the distal end 316 is provided in proximity in-situ to a region of interest in a body, for example. A plurality of ultrasound transducers 501₁-501_{N} each generates respective ultrasound beams (beams 1-beam N) in a scan across the region of interest. As shown in FIG. 5B, frame trigger (e.g., First Frame) provided at the beginning of a scan results in scanning over the region of interest and provides an image frame. As is known, the scanning may be sequential from ultrasound transducer 501₁ through 501_{N}, and at the next frame, the sequence is repeated to generate the next image frame (Frame 2). Moreover, the each ultrasound beam (beams 1-beam N) is triggered by a respective line trigger, with each successive beam being terminated at the reception of the next line trigger.

As depicted in FIGS. 5A and 5B, a first frame scan (Frame 1) begins with a frame trigger, with the first ultrasound transducer 501₁ being excited at the first line trigger (Line 1). Next, the second ultrasound transducer 502 is excited at the second line trigger (Line 2). As noted above this sequence continues until the end of the first frame at which point the second frame scan (Frame 2) begins with the second frame trigger, which coincides with the first line trigger of the second/next frame. The sequence begins anew by the exciting of the first ultrasound transducer 501₁ at the first line trigger (Line 1); followed by the second ultrasound transducer 502, which is excited at the second line trigger (not shown) of the second frame; and so forth until the termination of the second frame. As can be seen in FIGS. 5A and 5B, a signal (e.g., ultrasound signal 308, see FIG. 3) is received at the sensor 315 at a time coinciding with the line trigger n+1, with a maximum amplitude being received at a time At along the line n+1. This signal is used to determine the location of the sensor 315 relative to the first frame, and is superposed on the image of the frame at the particular time of its receipt, and thereby at a particular coordinate (x,y) of the coordinate system of the first frame image (e.g., composite image 218, comprising the image of the frame from the ultrasound imaging probe 211 and the superposed position 219 of the sensor).

In a representative embodiment, and as noted above, the position of the sensor 315 in the coordinate system of the first frame is determined at the processor of the console/control unit (e.g., processor 205). The sensor 315 transmits electrical signal 309, which is received at the sensing electrode(s) 320, 321, and provided to the processor 205 via a dedicated channel. These data are provided to the processor (e.g., processor 205), and the instructions stored in memory (e.g., memory 206) are executed by the processor to determine a position of the sensor 315 in an image frame, and to overlay the position of the sensor 315, and thus the distal end of the medical device 300 relative to the image of the first frame.

As can be appreciated, because the timing of the frame and line triggers are transmitted by the same clock 208, by measuring the time of receipt of the signal from the sensor 315 (likely the time of its peak magnitude), the location of the sensor 315 relative to the location of transducers of the transducer array (and thus the frame image) can be determined by straight forward velocity/time calculations. Notably, and as will be appreciated by one of ordinary skill in the art, since the electrical signal 309 travels slower in human tissue as compared to electrical conductors, the electrical signal 309 received at the sensing electrode(s) 320, 321 have a slight delay (less than 1 microsecond), which is accounted for by the instructions stored in the memory 206 executed by the processor 205. However, since electrical signals still travel more than a thousand times faster than ultrasound in human tissues, the electrical signal 309 can be detected well before ultrasound echoes, or from a region ultrasound echoes are too weak to be detected.

In the present representative embodiment, the x,y coordinates of the sensor 315 are known based on the timing of the return RF signal with respect to the transmit ultrasound waves. As such, the x,y coordinates of the sensor 315 are known relative to the n+1 transducer, the location of which is mapped to a coordinate system of the resultant first frame image. As such, the processor 205 of the console/control unit determines the position of the sensor 315, and superposes the position 219 on the frame image 218 by executing instructions stored in the memory.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An apparatus for performing a medical procedure, comprising:
a sensor adapted to be disposed in a body, and to convert an ultrasonic signal incident thereon into an electrical signal, the sensor comprising a lower electrode and an upper electrode, wherein the upper electrode is adapted to transmit the electrical signal wirelessly to an electrode of an ultrasound imaging probe, which is adapted to be disposed on a surface of the body.

2. The apparatus of claim 1, wherein the sensor is disposed at an end of the apparatus.

3. (canceled)

4. The apparatus of claim 1, wherein the electrical signal has a frequency selected at which the electrical reactance of a medium through which the electrical signal travels is less than approximately 100 Ohms.

5. The apparatus of claim 1, wherein the electrical reactance is in the range of approximately to 0 Ohms to 100 Ohms.

6. The apparatus of claim 1, wherein the lower electrode is a part of the apparatus, and the upper electrode is a part of the sensor.

7. The apparatus of claim 1, wherein the sensor comprises a piezoelectric element disposed between the lower and upper electrodes.

8. The apparatus of claim 7, wherein the piezoelectric element comprises a film piezoelectric material.

9. The apparatus of claim 7, wherein the piezoelectric element comprises a piezoceramic material.

10. The apparatus of claim 7, wherein the lower electrode is disposed between the piezoelectric element and the apparatus.

11. An ultrasound system, comprising:
an ultrasound imaging probe adapted to be disposed on a surface of the body, and to insonify a region of interest;
an apparatus configured to perform a medical procedure, the apparatus comprising: a sensor adapted to be disposed in a body to convert an ultrasonic signal incident thereon into an electrical signal; the sensor comprising a lower electrode and an upper electrode, wherein the upper electrode is adapted to wirelessly transmit the electrical signal to an electrode of the ultrasound imaging probe; and
a control unit remote from the ultrasound imaging probe and apparatus, the control unit being adapted to provide an image from the ultrasound imaging probe, the control unit comprising: a processor adapted overlay the a position of the apparatus on the image.

12. The ultrasound system of claim 11, the control unit comprising: a processor adapted to determine a location of the sensor relative to an image of a frame, and to overlay the location of the sensor on the image of the frame in a coordinate system of the frame.

13. The ultrasound system of claim 12, wherein the electrode of the ultrasound imaging probe provides an electrical signal from the electrode of the ultrasound imaging probe to the processor for the determination of the location of the sensor.

14. The ultrasound system of claim 11, wherein the electrode of the ultrasound imaging probe is connected to a channel separate from channels of the ultrasound imaging probe.

15. The ultrasound system of claim 12, wherein the control unit further comprises a clock configured to generate a clock signal, and the control unit is adapted to provide a trigger signal to the ultrasound imaging probe to commence an ultrasound scan over a frame.

16. The ultrasound system of claim 15, wherein the trigger signal is a frame trigger signal, and the clock is further configured to provide a line trigger signal, the control unit being configured to provide the frame trigger signal and the line trigger signal to the ultrasound imaging probe.
